(19) United States
(12) Patent Application Publication
(10) Pub. No.: US 2008/0262113 A1
(43) Pub. Date: Oct. 23, 2008

(54) PROCESS FOR MAKING AN EGG SHELL FT CATALYST

(76) Inventors: Hans Peter Alexander Calis, The Hague (NL); Marinus Johannes Reynhout, Amsterdam (NL); Guy Lode Magda Maria Verbist, Amsterdam (NL)

Correspondence Address: SHELL OIL COMPANY, P O BOX 2463, HOUSTON, TX, 772522463, US

(21) Appl. No.: 12/100,301

(22) Filed: Apr. 9, 2008

(30) Foreign Application Priority Data
Apr. 10, 2007 (EP) 0710585536

(51) Int. Cl. B01J 38/06; C07C 27/06; B01J 21/06; B01J 23/745; B01J 23/75; B01J 23/755

(52) U.S. Cl. 518/715; 502/300; 502/337; 502/338; 502/325; 502/335; 502/336; 502/332; 502/259; 502/260; 502/258; 502/55

(57) ABSTRACT

A process for preparing a Fischer-Tropsch catalyst comprising the steps of a) providing a particle comprising a support and having a catalytically active metal homogenously distributed therein, wherein at least 50 wt % of the catalytically active metal is present as divalent oxide or divalent hydroxide; b) treating the particle with a water vapour comprising gas having a relative humidity of at least 80% or with liquid water for at least two hours; and c) drying the catalyst particle.

This application claims the benefit of European Application No. 07105855.6 filed Apr. 10, 2007.

### FIELD OF THE INVENTION

The present invention relates to a process for preparing a catalyst or catalyst precursor, the obtained catalyst or catalyst precursor, and the use thereof in a Fischer-Tropsch process. More specifically, this invention relates to the preparation of Fischer-Tropsch catalysts and catalyst precursors comprising a catalytically active metal on a support, wherein the support is in the form of particles, and the catalytically active metal is predominantly present in the outer shell of the support particles, based on a precursor in which all ingredients were homogeneously distributed. A support for a catalyst is also referred to as carrier. Catalysts particles having a higher concentration of catalytically active metal in the outer shell than in the rest of the particle are sometimes referred to as egg shell catalyst particles.

### BACKGROUND OF THE INVENTION

The Fischer-Tropsch (FT) process involves the conversion of synthesis gas, a mixture comprising CO and H₂ which is sometimes referred to as syngas, to hydrocarbons. The FT process is in use for the manufacture of liquid hydrocarbons from other energy carriers, such as natural gas, coal, or biomass.

The FT process requires a catalyst, which in most cases comprises a catalytically active metal and a support. The catalytically active metal is often Co or Fe. The support is often a porous refractory oxide, such as silica, alumina, or titania.

In most cases the purpose of the FT process is to manufacture hydrocarbons having 5 or more carbon atoms. Methane is an unavoidable, but undesirable, by-product. It is desirable to define process conditions and develop FT catalysts that provide a low methane selectivity. High reaction temperatures tend to promote CO conversion, but at the same time increase methane selectivity. It is desirable to provide FT catalysts having a high activity, providing a high CO conversion at relatively low reaction temperatures, as one way of decreasing the methane selectivity.

It has been demonstrated that catalytic sites located deeply within the small pores of a catalyst particle tend to contribute to a high methane selectivity. The reason would be that the relative diffusion rates of H₂ on one hand, and CO on the other, favour the formation of methane. Put simply, H₂ is more likely to diffuse in a deep, narrow pore than a CO molecule is to diffuse into this pore, making it statistically more likely that the chain build-up will be terminated at C=1. Thus, the methane selectivity of a particulate FT catalyst may be decreased by locating the catalytic sites predominantly near the outer surface of the particle.

U.S. Pat. No. 4,962,078, issued Oct. 9, 1990 to Behrmann et al., discloses a supported particulate cobalt catalyst formed by dispersing cobalt as a thin catalytically active film upon a particulate titania or titania-containing support. The catalysts may be prepared by spraying a solution of a cobalt compound onto preheated titania or titania-containing particles. The particles are kept at a temperature of 140° C. or higher during spraying.

U.S. Pat. No. 4,977,126, issued Dec. 11, 1990 to Mauldin et al, discloses a process for the preparation of catalysts wherein a catalytically effective amount of cobalt is impregnated and dispersed as a film, or layer, on the peripheral outer surface of a particulate porous inorganic oxide support. The catalysts are prepared by spraying a bed of fluidized particulate support particles with a liquid containing a dispersed or dissolved cobalt metal compound. The bed is kept at a temperature of 50 to 100° C. during spraying.

For these spraying processes to provide good results it is necessary that the solvent stays with the cobalt compounds long enough to permit the liquid to be evenly distributed among the support particles, but not so long as to permit excessive diffusion of the cobalt compound into the pores of the support particles. It will be difficult to consistently find the right operating window for these two competing requirements.

U.S. Pat. No. 5,036,032, issued Jul. 30, 1991 to Iglesia et al., discloses the preparation of a so-called rim type FT catalyst whereby support particles are impregnated with a molten cobalt compound, such as cobalt nitrate. The temperature of the melt is kept near enough to the melting point to ensure a high viscosity of the melt. Due to the high viscosity diffusion of the melt into the pores of the support particles is minimized.

This process requires a tight control of the viscosity of the melt, and for the temperature to be adjusted to compensate for fluctuations in the composition of the cobalt compound, such as the presence of contaminants and crystal water, both of which may affect the viscosity of the melt. Further there are stringent requirements on porosity and pore size distribution for the carrier.

There is a need for a process for preparing egg shell catalyst particles not involving process parameters requiring such a tight control.

### SUMMARY OF THE INVENTION

The present invention relates to a process for preparing a Fischer-Tropsch catalyst or catalyst precursor comprising the steps of:

a) providing a catalyst or catalyst precursor particle comprising a support and having a catalytically active metal homogenously distributed therein, wherein at least 50 wt % of the catalytically active metal is present as divalent oxide or divalent hydroxide, calculated on the total weight of catalytically active metal atoms present in the particle;
b) treating the particle with a water vapour comprising gas having a relative humidity of at least 80% or with liquid water for at least two hours;
c) drying the catalyst particle; and
d) optionally subjecting the particle to hydrogen or a hydrogen comprising gas.

The support preferably comprises titania, alumina, silica, or mixtures thereof, titania being most preferred.

The catalyst or catalyst precursor may comprise one or more catalytically active metals. Preferably the catalyst or catalyst precursor comprises Co, Ni or Fe, or combinations thereof, Co being preferred. The catalyst or catalyst precursor may further comprise a promoter, preferably Mn or V.

The catalyst or catalyst precursor particle to be provided in step a) may be a fresh prepared particle. This is elaborated on below.

Another particle suitable to be provided in step a), and thus to be treated in step b), is a particle that has been used as a catalyst particle in a Fischer-Tropsch reaction. Such a particle may be referred to as a spent catalyst particle, used catalyst particle, or deactivated catalyst particle. The particle should comprise a support and should have the catalytically active metal homogenously distributed therein. And at least 50 wt % of the catalytically active metal should be present as divalent oxide or divalent hydroxide, calculated on the total weight of catalytically active metal atoms present in the particle. A spent catalyst may be treated with hydrogen or a hydrogen comprising gas to obtain the required amount of divalent oxide or divalent hydroxide. This is elaborated on below. In a preferred embodiment a spent catalyst is oxidized and in a later step treated with hydrogen or a hydrogen comprising gas.

During step (b) the particle is treated with water for at least two hours. When the temperature of the liquid water or steam is relatively high, the treatment period may be relatively short. When the temperature of the water is relatively low, the treatment period may be relatively long. The particle may be treated for several months. In most cases the treatment period does not have to be longer than two weeks. When the treatment has been performed long enough, the result of the treatment normally is that an egg shell catalyst or catalyst precursor particle is obtained.

During step (d) at least a part of the catalytically active metal present in the particle is reduced to its metal state.

Another aspect of the invention is a Fischer-Tropsch process wherein a catalyst is used that is prepared by the process of this invention.

### DETAILED DESCRIPTION OF THE INVENTION

A highly desirable aspect of the process of the present invention is that it involves to the most part conventional techniques and equipment.

Another advantage of the present invention is that with this process an egg shell catalyst or catalyst precursor can be obtained. Additionally, by means of a process comprising the process steps of the current invention a catalyst can be obtained that shows a relatively high activity. Further, by means of a process comprising the process steps of the current invention a catalyst can be obtained that show a relatively low methane selectivity.

One advantage is that egg shell catalyst or catalyst precursor particles can be prepared by treating particles that have been prepared by extruding a mixture comprising support material and catalytically active metal. This is very attractive because by this method the amount of catalytically active metal in the egg shell particles can be easily controlled.

A catalyst or catalyst precursor particle having a catalytically active metal homogenously distributed therein can be prepared with conventional techniques and equipment. It will be understood that the catalytically active metal will be present within the pores of the particles, which themselves are not necessarily homogenously distributed within the particle. The expression “a catalyst or catalyst precursor particle having a catalytically active metal homogenously distributed therein” means that it was prepared without any specific measures to create a bias toward deposition of the catalytically active metal predominantly either within the core or near the peripheral surface of the particle.

In case of a particle having a size between 1 and 6 mm, the amount of catalytically active metal close to the surface of the particle down to, for example 10 micrometers into the particle, preferably does not differ more than 5% absolute, more preferably not more than 1 to 2% absolute, from the amount of catalytically active metal in the bulk. For example, when the total amount of catalytically active metal is 20 wt %, calculated as the metal on the total weight of the particle, the amount of catalytically active metal within the particle preferably is 20±5 wt % for each 10 μm³, more preferably 20±2 wt % for each 10 μm³, regardless whether the sample is taken at the surface, in the bulk, or in the core of the particle.

The surface composition of the catalyst particles may be determined by visual inspection of the images obtained with a scanning electron microscope (SEM) in back scatter mode. A more quantitative assessment may be made by EDX (energy dispersive X-ray analysis). For this purpose catalyst particles are embedded in a resin. Embedded particles may be cut with a microtome so as to reveal their cores. Metal particles are visible in SEM in back scatter mode as light (or white) crystals against a darker grey background of the support material. EDX provides quantitative composition measurements of the surface layers of the particle.

A preferred catalyst or catalyst precursor comprises titania and cobalt.

A catalyst or catalyst precursor particle having a catalytically active metal homogenously distributed therein may be prepared using any conventional process for depositing a catalytically active metal onto a catalyst support. Suitable methods include impregnation, incipient wetness impregnation, ion exchange, mulling of catalytically active metal and support, and the like. Spraying of a solution of the catalytically active metal onto particles of the support material is also a useful method, with the understanding that it is not necessary to prevent the solution from diffusing into the pores of the support material. Thus, it is not necessary to preheat the support particles, or to choose a particular concentration of the catalytically active metal solution.

In the case of impregnation, any suitable solvent may be used for dissolving the catalytically active metal or a compound comprising the catalytically active metal. In most cases the catalytically active metal will be in the form of a salt. Nitrates and carboxylates are often preferred, as the anions can easily be removed by heating the catalyst particle in an oxygen containing gas, such as air. The solvent can be any solvent capable of dissolving the metal compound. Water is preferred in most cases because of its ease of handling and low cost.

Preferred methods for preparing a catalyst or catalyst precursor particle having a catalytically active metal homogenously distributed therein comprise a step in which the support material and the catalytically active metal or a compound comprising the catalytically active metal are mixed and/or mulled before the particle is formed. Preferred methods for forming the particle are pelleting and extrusion. Most preferably a mixture comprising the support material and the catalytically active metal or a compound comprising the catalytically active metal is extruded.

FT catalysts or catalyst precursors preferably comprise Fe, Ni and/or Co as the catalytically active metal, with Fe and/or Co being preferred, and with Co being the most preferred. However, it will be recognized that the present process is useful for any supported metal catalysts or catalyst precursors comprising a catalytically active metal that can be converted to a compound that is mobile on the support surface, as explained in more detail below.

Typically, the amount of catalytically active metal, calculated as the metal, present in the catalyst or catalyst precursor may range from 1 to 100 parts by weight per 100 parts by weight of support material, preferably from 3 to 50 parts by weight per 100 parts by weight of support material.

In addition to the catalytically active metal, the catalyst or catalyst precursor may further comprise a promoter. Suitable promoters include rhenium, zirconium, hafnium, cerium, thorium, uranium, vanadium, and manganese, with manganese and vanadium being preferred promoters, manganese most preferred. The catalytically active metal/promoter weight ratio is not critical and may range from 30:1 to 2:1, preferably from 20:1 to 5:1, calculated as the metal.

As discussed above, the catalyst or catalyst precursor particle that is subjected to the process of the present invention may have been prepared by any suitable method. In case, for example, the particle is prepared by means of impregnation of the catalytically active metal into the support, the promoter may be conveniently added by mixing a solution of a compound of the promoter, for example the nitrate salt, with a solution of a compound of the catalytically active metal in the same solvent, and contacting the support particles with the mixed solution. In case, for example, the particle is prepared by means of extruding a mixture comprising the support material and the catalytically active metal or a compound comprising the catalytically active metal, the promoter may be added to the mixture before extrusion.

Suitable support materials include alumina, silica, titania, and titania-containing materials, such as titania-alumina. Titania is the preferred support for FT catalysts. The support particles may be spherical, as for example obtained by spray-drying techniques, or they may be in a form as is commonly obtained by extrusion. Suitable support materials are those having a specific surface area, as measured by the B.E.T. method, in the range of 20 to 100 m²/g, and pore volumes, as measured for example with mercury intrusion techniques, in the range of 0.1 to 0.5 ml/g.

If the support material is in the form of a fine powder, the impregnated catalyst or catalyst precursor particles may be shaped into shaped particles, such as pellets or extrudates. After shaping, the particles may be calcined.

Preferably, the catalyst or catalyst precursor particle having a catalytically active metal homogenously distributed therein which is subjected to the process of the current invention has a size of at least 1 mm. Particles having a particle size of at least 1 mm are defined as particles having a longest internal straight length of at least 1 mm. The particle preferably has a size smaller than 6 mm. Most preferably the particle has a size in the range of 3 to 5 mm.

A highly suitable process for preparing a catalyst or catalyst precursor particle having the catalytically active metal homogenously distributed therein comprises the steps of:

(i) dispersing or co-mulling a support material and a catalytically active metal or a compound comprising a catalytically active metal;
(ii) shaping the dispersed or co-mulled material into a particle, preferably by means of extrusion;
(iii) optionally drying and/or calcining the particle at 400 to 600° C.

Alternatively the catalytically active metal may be deposited on pre-formed shaped support particles. After the catalytically active metal is deposited onto the support particles by any one of the common techniques, the catalyst particles may be air dried to remove excess solvent, such as water. The drying step could be carried out at ambient temperature, or at an increased temperature. Drying temperatures of up to 120° C. are suitable. Thereafter the catalyst particles may be dried and/or calcined at 400 to 600° C. During calcination Co₃O₄ will be formed in case the catalytically active metal is cobalt.

Alternatively, a spent catalyst particle having the catalytically active metal homogeneously distributed therein may be provided. Optionally the spent catalyst is oxidized at a temperature ranging from 200 to 400° C.

In a next, optional, step the catalyst or catalyst precursor particle may be subjected to a treatment with hydrogen or a hydrogen comprising gas. The purpose of this step is to bring the catalytically active metal into what will be referred to herein as its “sensible state”. This may also be referred to as a mild reduction step. In the case of Co its sensible state is Co²⁺. The reduction step is performed such that after this treatment with hydrogen or a hydrogen comprising gas a part of the catalytically active metal is present as divalent oxide or divalent hydroxide. Preferably at least 50%, more preferably at least 60%, even more preferably at least 70%, most preferably at least 80% of the catalytically active metal is present in divalent oxide or divalent hydroxide. The percentage is calculated as the amount of catalytically active metal atoms in its sensible state on the total amount of catalytically active metal atoms in the particle.

The amount of catalytically active metal present as divalent oxide or divalent hydroxide can be quantitatively determined by analysing one or more catalyst or catalyst precursor particles with X-ray diffraction (XRD). Alternatively, the amount of catalytically active metal present as divalent oxide or divalent hydroxide can be quantitatively determined by measuring during a reduction step the amount of water formed.

It is recommended to prevent or to minimize the reduction from proceeding to the metallic state. Water that is formed during this mild reduction step promotes the formation of the divalent oxide or hydroxide and suppresses the reduction to the metallic state, provided the reduction temperature is kept low. The conversion to the divalent oxide or hydroxide is more easily controlled if steam is added. Steam may be added to the hydrogen or hydrogen comprising gas. Additionally or alternatively, steam may be added before and/or during the reduction step separate from the hydrogen or hydrogen comprising gas. The reduction time ranges from 2 hours to 2 days, depending on the actual reduction temperature.

If the reduction is carried out without added steam, the reduction temperature is preferably in the range of 150 to 250° C. The partial hydrogen pressure preferably is in the range of 0.1-100 bar, more preferably in the range of 1-10 bar.

If steam is present a somewhat higher reduction temperature may be employed; the reduction temperature is preferably in the range of 150 to 300° C. In a preferred embodiment the mild reduction is carried out with a partial water pressure of 10³ to 106 Pa. The ratio of the hydrogen partial pressure and the water partial pressure may range from 0.01 to 10, with a ratio in the range of from 0.02 to 0.2 being preferred. In an alternate embodiment the catalyst may be reduced while immersed in liquid water, by bubbling hydrogen gas through the water seat.

It is believed that, after this mild reduction step, a catalytically active metal in its sensitive state, such as Co²⁺, will mostly be present in the catalyst particle as either an oxide or a hydroxide, or a mixture thereof.

During the mild reduction step CoO or Co(OH)₂ or a mixture thereof will be formed in case the catalyst or catalyst precursor particle comprises cobalt as catalytically active metal. The catalytically active metal present as divalent oxide will probably convert to divalent hydroxide upon contact with liquid water or with a water vapour comprising gas having a relative humidity of at least 80%. For example, CoO present in a catalyst particle will probably convert to Co(OH)₂ upon contact with liquid water or with a water vapour comprising gas having a relative humidity of at least 80%. The cobalt hydroxide is believed to be highly mobile in the pores of the support material, especially when the support is titania.

A highly suitable process for preparing a catalyst or catalyst precursor particle having the catalytically active metal homogenously distributed therein, whereby at least 50 wt % of the catalytically active metal is present as divalent oxide or divalent hydroxide, calculated on the total weight of catalytically active metal atoms present in the particle, comprises the steps of:

(i) dispersing or co-mulling a support material and a catalytically active metal or a compound comprising a catalytically active metal, whereby the support material preferably is titania and whereby the catalytically active material preferably is cobalt;
(ii) shaping the dispersed or co-mulled material into a particle, preferably by means of extrusion;
(iii) optionally drying and/or calcining the particle at 400 to 600° C.;
(iv) optionally mild reduction of the catalytically active metal with hydrogen or a hydrogen comprising gas.

It will be appreciated that a mild reduction step is needed when most of the catalytically active metal is present as Co₃O₄, for example after calcination in air.

The mild reduction step may be omitted if the catalytically active metal is incorporated in the support in this sensible state, and kept in this sensible state by omitting the customary calcination step. For example, a catalyst according to the present invention may be prepared by dry mixing titania and Co(OH)₂, adding water, kneading the mixture and shaping it into particles. After drying at a relatively low temperature, the cobalt in the particles will be mainly present as Co(OH)₂, and the particles may be treated with a gas having a relative humidity of at least 80%, or with liquid water, to form egg shell catalyst particles.

Another highly suitable process for preparing a catalyst or catalyst precursor particle having the catalytically active metal homogenously distributed therein, whereby at least 50 wt % of the catalytically active metal is present as divalent oxide or divalent hydroxide, calculated on the total weight of catalytically active metal atoms present in the particle, comprises the steps of:

(i) providing a particle that has been used as a catalyst particle in a Fischer-Tropsch reaction and that has the catalytically active metal homogenously distributed therein;
(ii) oxidizing the particle at a temperature ranging from 200 to 400° C.
(iii) mild reduction of the catalytically active metal with hydrogen or a hydrogen comprising gas.

In step b) of the process of the invention, a catalyst or catalyst precursor particle having a catalytically active metal homogenously distributed therein, whereby at least 50 wt % of the catalytically active metal is present as divalent oxide or divalent hydroxide, calculated on the total weight of catalytically active metal atoms present in the particle, is treated with water.

The water treatment may be performed with a gas mixture comprising steam such that the relative humidity is at least 80%. Preferably, however, the catalyst particle is treated with liquid water.

It has surprisingly been found that a water treatment results in a migration of the catalytically active metal towards the peripheral surface of the catalyst particle. As a result of this migration the outer shell of the particle becomes enriched in catalytically active metal, whereas the core of the particle becomes depleted in the catalytically active metal. The terms “enriched” and “depleted” are to be understood with reference to the overall composition of the catalyst particle.

The time required for the water treatment step depends on the temperature of the water or steam. At room temperature the water treatment step suitably ranges from up to a day to several days or more than a week. At a higher temperature, for example 80° C., the treatment time may be kept shorter, e.g. in the range of 2 hours to one or 2 days. The temperature of the water or steam preferably is 0° C. or higher, more preferably 10° C. or higher, even more preferably 20° C. or higher. The temperature of the water or steam preferably is 273° C. or lower, more preferably 150° C. or lower, even more preferably 100° C. or lower.

After the water treatment the catalyst particles may be dried by any suitable technique. After drying, the composition of the catalyst particles may be determined, for example using SEM and/or EDX, as described above. The composition after treatment can then be compared to the composition determined before treatment.

After drying the “water treated” catalyst or catalyst precursor particle and before use of it in a Fischer-Tropsch process, the particle may be subjected to a reduction step. This may be performed using hydrogen or a hydrogen containing gas. The temperature during the reduction preferably is in the range of 180 to 400° C., more preferably in the range of 200 to 350° C. During the reduction a part of the catalytically active metal is reduced to its metal state. After this reduction, or before starting Fischer-Tropsch synthesis, preferably at least 70%, more preferably at least 80%, of the catalytically active metal is present in its metal state. The percentage is calculated as the amount of catalytically active metal in its metal state on the total amount of catalytically active metal atoms in the particle.

### EXAMPLE 1

### Sample Preparation

Titania particles available from a commercial source (P25 from Degussa) were mixed with Co(OH)₂ and Mn(OH)₂. The respective amounts of titania, cobalt hydroxide and manganese hydroxide were calculated to result in a catalyst composition comprising 20 wt % Co and 1.2 wt % Mn, both calculated as the metal.

Enough water was added to form a kneadable paste. The paste was kneaded and extruded into 1.7 mm trilobes. The resulting trilobe shaped particles were dried at 120° C., then calcined in air for 3 hours at 550° C.

The resulting catalyst particles had a nominal composition of 20 wt % Co, 1.2 wt % Mn, both calculated as the metal, the balance being titania. The nominal composition of catalyst particles may be determined by dissolving the particles in nitric acid, and determining the amount of cobalt and manganese.

Both the Co and the Mn were homogenously distributed throughout the shaped catalyst particles. With SEM/EDX was determined that throughout the particles the concentration of the cobalt was 20±2 wt %.

### EXAMPLE 2

### Mild Reduction

Catalyst samples were prepared as in Example 1. Five samples were subjected to wet reduction at different hydrogen/steam ratios.

The aim was to examine the effect of steam during mild reduction when preparing catalyst particles with at least 50 wt % of the cobalt in the form of CoO or Co(OH)₂ from catalyst particles prepared as in Example 1. The quality of the mildly reduced samples was determined by inspecting them visually and by determining their activity. The samples were thus inspected directly after the mild reduction step; they were not treated with water.

After the mild reduction, the samples were visually inspected in SEM/TEM for the presence of large Co clusters. Preferably there are no or hardly any large cobalt clusters. Additionally, the catalytic activities were measured in a model Fischer-Tropsch reactor. The catalytic activities measured were expressed as an activity factor (an activity factor of 1 being STY=100 g/gkg.hr at 200° C.). The results are summarized in Table 1.

TABLE 1

| | Sample # | | | | |
|---|---|---|---|---|---|
| Parameter | 1 | 2 | 3 | 4 | 5 |
| Average temp. during | 268 | 274 | 260 | 270 | 270 |
| wet reduction [° C.] | | | | | |
| Average pressure during | 6.6 | 5.9 | 5.9 | 6.7 | 6.7 |
| wet reduction [bara] | | | | | |
| Average water vapour | 15 | 10 | 4.3 | 23.7 | 23.7 |
| concentration during | | | | | |
| first 20 hrs. of wet | | | | | |
| reduction [% v] | | | | | |
| Average water vapour | 20 | 7.2 | 3.7 | 23.7 | 23.7 |
| concentration during | | | | | |
| first night | | | | | |
| Activity factor | 0.35 | 0.68 | 0.91 | 0.36 | 0.36 |
| Large Co clusters | Yes | No | No | Yes | Yes |
| (SEM/TEM) | | | | | |

From these results it appears that for these samples a water vapour concentration during wet reduction of more than 10% v tends to result in large Co clusters at the surface of the catalyst particles. Correspondingly the activity factor is reduced.

Hence, catalyst particles having cobalt homogenously distributed therein, whereby at least 50 wt % of the cobalt is present as divalent oxide or divalent hydroxide, calculated on the total weight of cobalt atoms present in the particle, may, for example, be prepared by:
an extrusion process as exemplified in Example 1; followed by
a mild reduction step as exemplified in Example 2 with a water vapour concentration during wet reduction of less than 10% v.

### EXAMPLE 3

### Water Treatment According to Invention

Catalyst particles prepared as in Example 1 were reduced in a mixture of hydrogen (partial pressure 6.105 Pa) containing 3% steam for 24 hours at 260° C. After this mild reduction the catalyst particles were treated with liquid water at room temperature for 7 days. Subsequently the catalyst particles were dried in air at 120° C.

After the water treatment the catalyst particles still had a nominal composition of 20 wt % Co, 1.2 wt % Mn, both calculated as the metal, the balance being titania.

The water treated catalyst particles had a composition near the surface (down to 10 μm from the surface) of 34 wt % Co, 14 wt % Mn (both calculated as the metal), the balance being titania. In the center the particles had a composition of 17 wt % Co and 1.0 wt % Mn.

After a reduction step in which of most of the cobalt was reduced to its metal state, the catalyst particles were used in a FT reaction under normal reaction conditions. A mixture of hydrocarbons was formed, in particular linear alkanes and olefins having 5 or more carbon atoms. The reaction had a favourably low methane selectivity.

What is claimed is:

1. A process for preparing a Fischer-Tropsch catalyst or catalyst precursor particle comprising the steps of:
a) providing a catalyst or catalyst precursor particle comprising a support and having a catalytically active metal homogenously distributed therein, wherein at least 50 wt % of the catalytically active metal is present as divalent oxide or divalent hydroxide, calculated on the total weight of catalytically active metal atoms present in the particle;
b) treating the particle with a water vapour comprising gas having a relative humidity of at least 80% or with liquid water for at least two hours;
c) drying the catalyst particle; and
d) optionally subjecting the particle to contact with hydrogen or a hydrogen comprising gas.

2. The process of claim 1 wherein step a) comprises the steps of:
(i) providing a catalyst or catalyst precursor particle having the catalytically active metal homogenously distributed therein, wherein the particle comprises a fresh prepared particle; and
(ii) mild reduction of the catalytically active metal with hydrogen or a hydrogen comprising gas.

3. The process of claim 2 wherein step (i) comprises the steps of:
(I) dispersing or co-mulling a support material and a catalytically active metal or a compound comprising a catalytically active metal;
(II) shaping the dispersed or co-mulled material into a particle, preferably by means of extrusion;
(III) optionally drying and/or calcining the particle at 400 to 600° C.

4. The process of claim 2 wherein, in step (ii), the particle is immersed in liquid water.

5. The process of claim 2 wherein, in step (ii), the particle is treated with a gas mixture comprising hydrogen and steam.

6. The process of claim 1 wherein the catalyst or catalyst precursor particle comprises Co, Ni, Fe or mixtures thereof as catalytically active metal.

7. The process of claim 1 wherein the support comprises alumina, silica, titania, or mixtures thereof.

8. The process of claim 1 wherein step a) comprises the steps of:
(i) providing a catalyst or catalyst precursor particle having the catalytically active metal homogeneously distributed therein, wherein the particle comprises a particle that has been used in a Fischer-Tropsch reaction; and
(ii) mild reduction of the catalytically active metal with hydrogen or a hydrogen comprising gas.

9. The process of claim 8 wherein step (i) comprises the steps of:
(I) providing a particle that has been used as catalyst particle in a Fischer-Tropsch reaction;
(II) oxidizing the particle at a temperature ranging from 200 to 400° C.

10. A catalyst or catalyst precursor as prepared by the process of claim 1.

11. A Fischer-Tropsch process comprising the step of passing a mixture of carbon monoxide and hydrogen over a catalyst prepared by the process of claim 1.
